(19) United States
(12) Patent Application Publication
(10) Pub. No.: US 2010/0021436 A1
(43) Pub. Date: Jan. 28, 2010

(54) MULTIPOTENT ADULT STEM CELL DERIVED FROM CANINE UMBILICAL CORD BLOOD, PLACENTA AND CANINE FETUS HEART, METHOD FOR PREPARING THE SAME AND CELLULAR THERAPEUTICS CONTAINING THE SAME

(75) Inventors: Kyung Sun Kang, Seoul (KR); Oh Kyung Kwon, Seoul (KR); Yun Hyeok Jeong, Gyeongsangbuk-do (KR); Ji Hey Lim, Seoul (KR); Chang Soo Jung, Seoul (KR)

Correspondence Address: MOORE & VAN ALLEN PLLC, P.O. BOX 13706, Research Triangle Park, NC, 27709, US

(73) Assignee: SEOUL NATIONAL UNIVERSITY INDUSTRY FOUNDATION, Seoul (KR)

(21) Appl. No.: 12/300,281

(22) PCT Filed: May 12, 2007
(86) PCT No.: PCT/KR2007/002354

§ 371 Date: Jun. 18, 2009

(30) Foreign Application Priority Data
May 12, 2006 (KR) 10-2006-0042850

(51) Int. Cl. A61K 35/12; C12N 5/074

(52) U.S. Cl. 424/93.7; 435/350

(57) ABSTRACT

The present invention relates to multipotent adult stem cells derived from canine umbilical cord blood, placental blood and blood sample from canine fetal heart, and a method for preparing the same as well as a cellular therapeutic agent containing the same, more specifically, to a multipotent adult stem cell isolated by culturing an eukaryotic cell derived from canine umbilical cord blood, placental blood and blood sample from canine fetal heart in a FBS-containing medium and a method for preparing the same. Adult stem cells according to the present invention are derived from canine umbilical cord blood, placental blood and blood sample from canine fetal heart. The adult stem cells have characteristics highly similar to human mesenchymal stem cells as well as remarkable cell growth at the initial step compared to human UCB-derived mesenchymal stem cells so that the cells are useful to treat canine incurable diseases and difficult-to-cure diseases. Furthermore, multipotent adult stem cells are effective to treat musculoskeletal diseases and neural diseases due to the ability to differentiate into osteogenic cells and neural cells.

### TECHNICAL FIELD

The present invention relates to a multipotent adult stem cell derived from canine umbilical cord blood, placental blood and canine fetal heart, and a method for preparing the same, more specifically, to a multipotent adult stem cell obtained by culturing eukaryotic cells derived from blood sample from canine fetal heart, and canine umbilical cord blood or placental blood, in a FBS-containing medium and a method for preparing the same.

### BACKGROUND ART

It has been recognized that totipotent stem cells having the ability to form all the organs by proliferation and differentiation can not only treat most diseases but also fundamentally heal organ injuries. Furthermore, it has been suggested that cell therapy using stem cells can be applied to the regeneration of most human organs and the treatment of incurable diseases including Parkinson's disease, various cancers, diabetes, and spinal cord injuries.

Cell therapy is a method for treating or preventing diseases by externally proliferating or selecting autologous stem cells, allogeneic stem cells or xenogeneic stem cells, or another method of changing biological properties of cells in order to restore the function of a malfunctioning cell or tissue. Cell therapy has infinite possibilities in the treatment of incurable and difficult-to-cure diseases since it has a very wide range of application areas, such as proliferating somatic cells collected from the patient himself, other persons, or other animals, or differentiating stem cells into desired cell types to use for the treatment of diseases.

Stem cells refer to cells having both self-replication ability and the ability to differentiate into at least two cells, and can be classified into totipotent stem cells, pluripotent stem cells, and multipotent stem cells.

Totipotent stem cells are cells having totipotent properties capable of developing into one perfect individual, and these properties are possessed by cells up to the 8-cell stage after the fertilization of an oocyte and a sperm. When these cells are isolated and transplanted into the uterus, they can develop into one perfect individual.

Pluripotent stem cells, which are cells capable of developing into various cells and tissues derived from the ectodermal, mesodermal and endodermal layers, are derived from an inner cell mass located inside of blastocysts at 4-5 days after fertilization. These cells are called “embryonic stem cells” and can differentiate into various other tissue cells but not form new living organisms.

Multipotent stem cells, which are stem cells capable of differentiating into only cells specific to tissues and organs containing these cells, are involved not only in the growth and development of various tissues and organs in the fetal, neonatal and adult periods but also in the maintenance of homeostasis of adult tissues and in function to trigger regeneration upon tissue damage. Tissue-specific multipotent cells are collectively called “adult stem cells”.

Adult stem cells are obtained by harvesting the preexisting cells from various human organs and developing the cells into stem cells, which are characterized by differentiating into only specific tissues. However, recently, it is spotlighted that experiments for differentiating adult stem cells into various tissues including liver cells etc., are successful.

The multipotent stem cells were first isolated from adult marrow (Jiang et al., Nature, 418:41, 2002), and then also found in other various adult tissues (Verfaillie, Trends Cell Biol., 12:502, 2002). In other words, although bone marrow is the most widely known source of stem cells, multipotent stem cells were also found in the skin, blood vessels, muscles and brains (Tomas et al., Nat. Cell Biol., 3:778, 2001; Sampaolesi et al., Science, 301:487, 2003; Jiang et al., Exp. Hematol., 30:896, 2002).

Furthermore, both hematopoietic and mesenchymal stem cells, isolated recently from human umbilical cord blood (UCB), in addition to bone marrow are induced to differentiate into various cell types so that there is high possibility of them being used as a cell therapeutic drug for the treatment of blood-related diseases, thereby increasing their significance as a source of supply to harvest adult stem cells

In general, hematopoietic stem cells are known to show positive responses to CD34 antibody against a surface antigen, whereas mesenchymal stem cells show negative reaction. According to the results of Flow Analysis Cell Sorting, the characteristic of CD34 (−) cells isolated from human bone marrow generally shows similar expression pattern of fluorescence-labeled antibodies to that of UCB-derived mesenchymal stem cells. It was found by differentiation experiments that the UCB-derived mesenchymal stem cells differentiate into various types of cells, suggesting the possibility to be used in studies related to differentiation and a variety of cellular therapies like mesenchymal stem cells from human bone marrow. However, up to now, a few mesenchymal stem cells from human umbilical cord blood exist at the initial culture step and thus it would be unavoidably limited in using the cells for analyses and differentiation experiments until securing enough number of cells.

Accordingly, the present inventors have isolated mesenchymal stem cells from canine umbilical cord blood and blood sample from canine fetal heart and cultured by the same method as the method of isolating the eukaryotic cell layer from human umbilical cord blood and culturing stem cells, and as a result, found that mesenchymal stem cells isolated from canine umbilical cord blood and blood sample from canine fetal heart show excellent cell growth at the initial culture step contrary to human mesenchymal stem cells and have highly similar characteristics to that of mesenchymal stem cells from human umbilical cord blood or bone marrow from the result of FACS analyses and cell differentiation experiments, thereby completing the present invention.

### SUMMARY OF THE INVENTION

A main object of the present invention is to provide a multipotent adult stem cell derived from canine umbilical cord blood, placental blood and blood sample from canine fetal heart, which have properties similar to a human mesenchymal stem cell as well as show remarkable cell growth at the initial culture step, and a method for preparing the same.

Another object of the present invention is to provide a method for differentiating the multipotent stem cells into cells of the musculoskeletal system and the cerebral nervous system, and a cellular therapeutic agent containing the differentiated cells or the adult stem cells.

In order to achieve the above objects, in one aspect, the present invention provides an adult stem cell and a method for producing the same, in which the adult stem cell is obtained by culturing eukaryotic cells derived from blood sample from canine fetal heart, and canine umbilical cord blood or placental blood, in FBS-containing medium and show the following characteristics of:
(a) showing positive immunological responses to one or more of antigens selected from the group consisting of MHC class I, CD44(BD) and CD90, and positive or negative immunological responses to CD34, and negative immunological responses to CD45, CD14, CD3, CD4, CD8, CD11c, CD172a and HLA-DR;
(b) growing adhered to plastic and showing spindle-shaped morphological feature; and
(c) having the ability to differentiate into the cells derived from endoderm, ectoderm, and mesoderm.

In another aspect, the present invention provides a cellular therapeutic agent for treating musculoskeletal diseases, a cellular therapeutic agent for treating neural diseases, and a cellular therapeutic agent for treating canine incurable diseases, which contain the adult stem cell as an active ingredient.

In still another aspect, the present invention provides a method for differentiating the adult stem cells into osteogenic cells, the method comprising mixing the adult stem cells with TCP (Trocalcium phosphate) and transplanting them orthotopically or heterotopically. Also, the present invention provides a cellular therapeutic agent for treating musculoskeletal diseases, which contains the osteogenic cells differentiated by the above-mentioned method as an active ingredient.

In still another aspect, the present invention provides a method for differentiating the adult stem cells into neural cells, the method comprising the steps of: (a) pre-incubating the adult stem cells in a DMEM medium containing β-mercaptoethanol; and (b) treating the pre-incubated broth with DMSO and BHA (butylated hydroxyanisole) so as to induce neural differentiation.

Another features and embodiments of the present invention will be more clarified from the following “detailed description” and the appended “claims”.

### BRIEF DESCRIPTION OF DRAWINGS

FIG. 1 is photographs taken by a microscope, showing multipotent adult stem cells derived from canine umbilical cord blood and blood sample from canine fetal heart according to the present invention (A: a photograph taken at 40× magnification, B: a photograph taken at 100× magnification, C: a photograph taken at 200× magnification, showing the morphology of cells 3 days after culturing mononuclear cells isolated canine umbilical cord blood and blood sample from canine fetal heart).

FIG. 2 shows the process of differentiation of the inventive adult stem cells derived from canine umbilical cord blood and blood sample from canine fetal heart into osteogenic cells in vitro (A: control, D, E and F: culturing in an osteogenic induction medium).

FIG. 3 presents osteogenic cells differentiated from the multipotent adult stem cells which are derived from canine umbilical cord blood and blood sample from canine fetal heart according to the present invention in vivo (A: a photograph taken of tissue 1 week after mixing the stem cells derived from canine umbilical cord blood and blood sample from canine fetal heart with TCP, B: a photograph taken of tissue 8 weeks after the mixing, C: image B at 400× magnification)

FIGS. 4˜8 illustrate that cells resulting from the differentiation of the multipotent adult stem cells according to the present invention into nerve cells, show positive expression of specific neural markers, GFAP (Glial Fibrillary Acidic Protein), MAP2 (Microtubule-Associated Protein2), and Tuj1. FIG. 4 shows images of the adult stem cells according to the present invention expressing GFAP primary antibody (A: cells expressing GFAP, B: Hoechst staining, C: merger of A and B, D: control). FIG. 5 shows images of the adult stem cells according to the present invention expressing MAP2 primary antibody (A: cells expressing MAP2, B: Hoechst staining, C: merger of A and B, D: control). FIG. 6 shows images of the adult stem cells according to the present invention expressing Tuj1 primary antibody (A: cells expressing Tuj1, B: Hoechst staining, C: merger of A and B, D: control). FIGS. 7 and 8 show images of a negative control in which a secondary antibody is reacted with cells without reaction with a primary antibody (A and E: cells which is reacted with a secondary antibody without reaction with a primary antibody, B and F: DIC images of a confocal microscope, C and G: Hoechst staining of the nuclei of cells, D: merger of A, B, and C, H: merger of E, F, and G).

FIG. 9 is a graph showing Olby scores of experiment groups at 2, 4, 16, and 32 weeks after transplantation of multipotent adult stem cells of the present invention (No. 1: experimental dog 1, No. 2: experimental dog 2, No. 3: experimental dog 3, No. 4: experimental dog 4)

FIG. 10 shows transverse T2-weighted images of the spinal cord lesion of experimental dogs where the canine UCB-derived multipotent adult stem cells were transplanted (A: before stem cell transplantation, B: after stem cell transplantation, Arrow: the area of spinal cord showing high signal intensity on the T2-weighted image, Arrow heads: the increased epaxial muscle).

### DETAILED DESCRIPTION OF THE INVENTION, AND PREFERRED EMBODIMENTS

The present invention, in one aspect, relates to an adult stem cell and a method for producing the same, in which the adult stem cell is obtained by culturing eukaryotic cells derived from blood sample from canine fetal heart, and canine umbilical cord blood or placental blood, in FBS-containing medium and show the following characteristics of:
(a) showing positive immunological responses to one or more of antigens selected from the group consisting of MHC class I, CD44(BD) and CD90, and positive or negative immunological responses to CD34, and negative immunological responses to CD45, CD14, CD3, CD4, CD8, CD11c, CD172a and HLA-DR;
(b) growing adhered to plastic and showing spindle-shaped morphological features; and
(c) having the ability to differentiate into the cells derived from endoderm, ectoderm, and mesoderm.

In the present invention, the medium is DMEM and preferably contains 1˜30% FBS and the adult stem cells preferably comprise having excellent cell growth at the initial culture step. Also, the ectoderm-derived cell is preferably an osteogenic cell but not limited thereto and any cell can be used as long as it is derived from ectoderm. In other aspect, the ectoderm-derived cell is preferably a nerve cell and the nerve cell is preferably a cerebral nerve cell.

In the present invention, multipotent adult stem cells were isolated from canine umbilical cord blood and blood sample from canine fetal heart. As a result of examining the culture characteristics of the isolated adult stem cells, it was found that the adult stem cells grew adhered to the flask bottom.

Generally, methods for obtaining multipotent stem cells include a FACS method using a flow cytometer with a cell sorting function (Int. Immunol., 10(3):275, 1998), a method using magnetic beads, and a panning method using an antibody specifically recognizing multipotent stem cells (J. Immunol., 141(8):2797, 1998). Also, methods for obtaining multipotent stem cells from a large amount of culture broth include a method in which antibodies specifically recognizing molecules expressed on the cell surface (hereinafter, referred to as “surface antigens”) are used alone or in combination as columns.

Flow cytometry sorting methods include a water drop charge method and a cell capture method and the like. In any of these methods, an antibody specifically recognizing an antigen on the cell surface is fluorescently labeled and the intensity of fluorescence from the labeled antigen-antibody complex is converted to an electric signal, thereby quantifying the amounts of the antigen expressed. It is also possible to separate cells expressing a plurality of surface antigens by combining types of fluorescence used. The fluorescent substance which is usable in this case include FITC (fluorescein isothiocyanate), PE (phycoerythrin), APC (allophycocyanin), TR (Texas Red), Cy 3, CyChrome, Red 613, Red 670, TRI-Color, Quantum Red, etc.

FACS methods using a flow cytometer include: a method where obtained stem cell broth is collected, from which cells are isolated by such as centrifugation, and stained directly with antibodies; and a method where the cells are cultured and proliferated in a suitable medium and then stained with antibodies. The staining of cells is performed by mixing a primary antibody recognizing a surface antigen with a target cell sample and incubating the mixture on ice for 30 minutes to 1 hour. When the primary antibody is fluorescently labeled, the cells are isolated with a flow cytometer after washing. When the primary antibody is not fluorescently labeled, cells reacted with the primary antibody and a fluorescent labeled secondary antibody having a binding activity specific for the primary antibody is mixed after washing, and incubated on ice water for 30 minutes to 1 hour. After washing, the cells stained with the primary and secondary antibodies are isolated with a flow cytometer.

Various surface antigens may include hematopoietic-associated antigens, surface antigens of mesenchymal cells, and antigens specific to nervous system neurons and the like. The hematopoietic-associated antigens include CD34, CD45, etc., the surface antigens of mesenchymal cells include SH-2, SH-3, etc., and the antigens specific to nervous system neurons include NSE, GFAP, etc. A desired cell can be obtained by using antibodies recognizing the above-described surface antigens, alone or in combination.

As a result of examining the immunological properties of the inventive isolated multipotent adult stem cells using a FACS method, the multipotent adult stem cells showed positive immunological responses to MHC class I, CD44 (BD) and CD90, and positive or negative immunological responses to CD34, and negative immunological responses to CD45, CD14, CD3, CD4, CD8, CD11c, CD172a and HLA-DR.

The stem cells according to the present invention are useful as cellular therapeutic agents because the stem cells are capable of differentiating into osteogenic cells and neural cells. Therefore, the present invention, in another aspect, relates to a cellular therapeutic agent for treating musculoskeletal diseases, a cellular therapeutic agent for treating neural diseases, and a cellular therapeutic agent for treating canine incurable diseases, which contains the adult stem cells as an active ingredient.

In another aspect, the present invention relates to a method for differentiating the adult stem cells into osteogenic cells, the method comprising mixing adult stem cells with TCP (Trocalcium phosphate) and transplanting them orthotopically or heterotopically. Also, the present invention provides a cellular therapeutic agent for treating musculoskeletal diseases, which contains the osteogenic cells differentiated by the above-mentioned method as an active ingredient.

In still another aspect, the present invention provides a method for differentiating the adult stem cells into neural cells, the method comprising the steps of: (a) pre-incubating the adult stem cells in a DMEM medium containing β-mercaptoethanol; and (b) treating the pre-incubated broth with DMSO and BHA (butylated hydroxyanisole) so as to induce neural differentiation.

### EXAMPLES

Hereinafter, the present invention will be described in more detail by examples. However, it is obvious to a person skilled in the art that these examples are for illustrative purpose only and are not construed to limit the scope of the present invention.

In the following examples, especially, adult stem cells derived from canine umbilical cord blood and blood sample from canine fetal heart were isolated for experiments, but it is not limited thereto, and it would also be useful to a person skilled in the art to apply adult stem cells derived from canine placental blood by isolating and proliferating them according to the present invention

### Example 1

### Isolation of Adult Stem Cells from Canine Umbilical Cord Blood (UCB) and Blood Sample from Canine Fetal Heart (FH) and the Culture Thereof

Canine umbilical cord blood and blood sample collected from canine fetal heart were diluted in PBS at a ratio of 1:1 to stir. Then, blood sample was laid over Ficoll-Pague at a ratio of 15:25 (Ficoll-Pague: Canine umbilical cord blood). the blood sample diluted in PBS at a ratio of 1:1 was spilled smoothly onto 15 ml of ficoll solution to cause layer separation, followed by centrifugation at 1500˜3500 rpm for 5˜30 minutes. After the centrifugation, thin buffy coat layer in the middle layer of a tube was formed and was transferred to a new tube using a micropipette. HBSS was added to the tube to make a tube containing 30 mL of solution, followed by centrifugation at 1500˜3000 rpm for 5˜20 minutes, from which the supernatant was completely removed and the precipitation solution was kept immediately on ice.

After adding 1 mL of HBSS to the precipitation solution and pipetting softly, 29 mL of HBSS was additionally added to mix uniformly by shaking the tube, followed by centrifugation at 1000˜2000 rpm for 5˜20 minutes. The supernatant was removed and the remnant was centrifugated again at 1000˜2000 rpm for 5˜20 minutes (repeating the above process twice). After the centrifugation, the supernatant was removed and the precipitation solution was suspended in 1 mL of DMEM to count cells.

After suspending cells in a medium, DMEM (low glucose+20% FBS) which is suitable for culturing cMSC (canine Mesenchymal Stem Cells)-like cells, the suspension was diluted at a concentration of 1˜2×10⁸ cells/20 mL medium in a T-75 flask. After culturing cells for 3 days in a CO₂ incubator, the supernatant was transferred into a new T-75 flask and culture broth containing ingredients equal to the broth used in the initial culture was poured onto the cells adhered to the flask bottom. 4˜10 days later, the cells were detached by trypsinization to be seeded at a concentration of 1×10³˜1×10⁵/mL in a new flask.

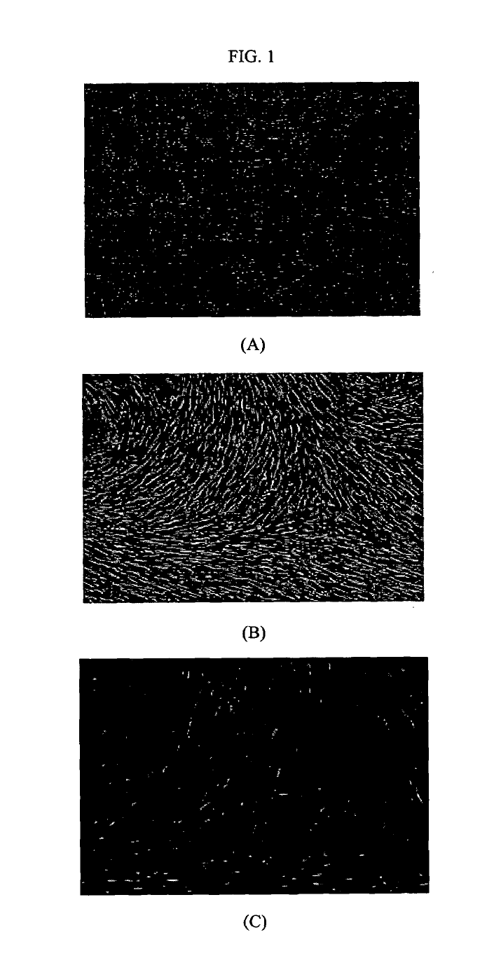

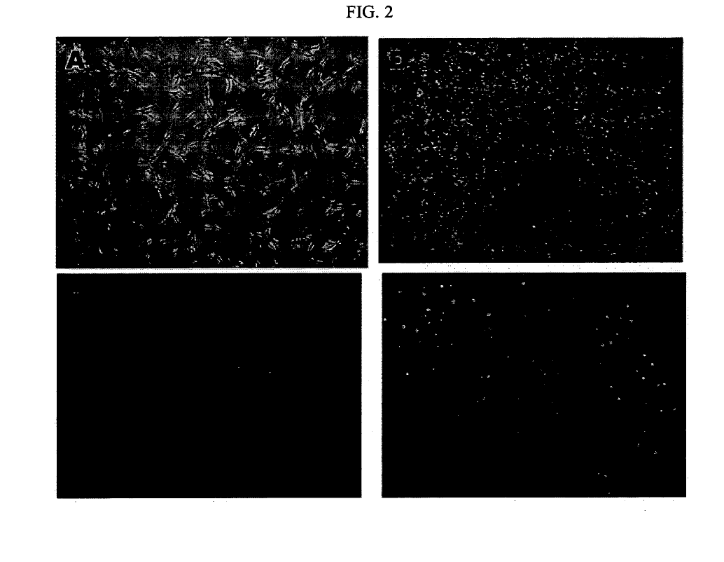

FIG. 1 shows the morphology of cells 3 days after culturing mononuclear cells isolated from canine umbilical cord blood, which is obtained by observation of multipotent adult stem cells derived from canine umbilical cord blood, placental blood and blood sample from canine fetal heart according to the present invention on a microscope. Fibroblast-like cells grew attached to a flask bottom 3˜7 days after the culture in the same manner as that of human UCB-derived mesenchymal stem cells.

### Example 2

### Immunological Characteristics of Multipotent Adult Stem Cells Derived from Canine Umbilical Cord Blood and Blood Sample from Canine Fetal Heart

The expression pattern of cell surface antigens was examined to determine immunological characteristics of multipotent adult stem cells prepared in Example 1.

P0 cells were collected after the primary culture and seeded into a new T-75 flask to culture P1 cells. The collected P1 cells were bound to primary antibodies against CD34, MHC Class I, CD44, CD90, CD14, CD45, CD3, CD4, CD8, CD172a, CD11c, HLA-DR and then were bound to fluorescent-labeled antibodies to carry out FACS analysis using indirect immunological labeling. As a result, adult stem cells according to the present invention showed the following immunological characteristics.

TABLE 1

| Antibody | Canine MSC from umbilical cord (%) |
|---|---|
| CD34 | 29.48 |
| MHC class 1 | 59.77 |
| CD44 | 89.81 |
| CD90 | 25.46 |
| CD14 | 0 |
| CD45 | 0 |
| CD3 | 0 |
| CD4 | 0 |
| CD8 | 0 |
| CD172a | 0 |
| CD11c | 0 |
| HLA-DR | 0 |

### Example 3

### Differentiation of Multipotent Adult Stem Cells Derived from Canine Umbilical Cord Blood and Blood Sample from Canine Fetal Heart into Osteogenic Cells

### (1) In Vitro Test

Multipotent adult stem cells derived from canine umbilical cord blood and blood sample from canine fetal heart, obtained in Example 1 were cultured for 30 days in an osteogenic induction medium containing 10% FBS, 10 mM β-glycerophosphate, 0.1 μM dexamethasone (Sigma-Aldrich), and 50 μM ascorbate. Osteogenic differentiation was measured by calcium mineralization. For Alizarian red S staining, the cells were washed twice with distilled water and fixed with 70% ice-cold solution for 1 hour. After carefully washing 7 times with distilled water and 2 times with distilled water at an ambient temperature, the cells were stained with 40 mM Alizarin Red S for 10 minutes.

5-times subcultured cells were maintained in an osteogenic induction medium so as to differentiate into osteocytes. The morphology of cells was changed 2 weeks after the differentiation induction. At this time, the supplementary medium was replaced once every 3 days. At 30 days after the induction, the cells were fixed with Alizarin Red S stain.

Consequently, as described in FIG. 2, the differentiated osteocytes could be found. In FIG. 2, (A) shows negative control cells cultured in a low glucose-DMEM medium with 20% FBS, 1% penicillin, and streptomycin and D, E, F show the cells cultured in the osteogenic induction medium.

### (2) In Vivo Test

After the primary culture of multipotent adult stem cell broth derived from canine umbilical cord blood and blood sample from canine fetal heart obtained in Example 1 to collect P2 cells, the collected P2 cells were mixed with beta-TCP (tricalcium phosphate) and transplanted heterotopically into canine subcutaneous tissue. At 1, 4, 8 weeks after transplanting, the transplant site was biopsied and treated, followed by hematoxylin-eosin (H&E) staining.

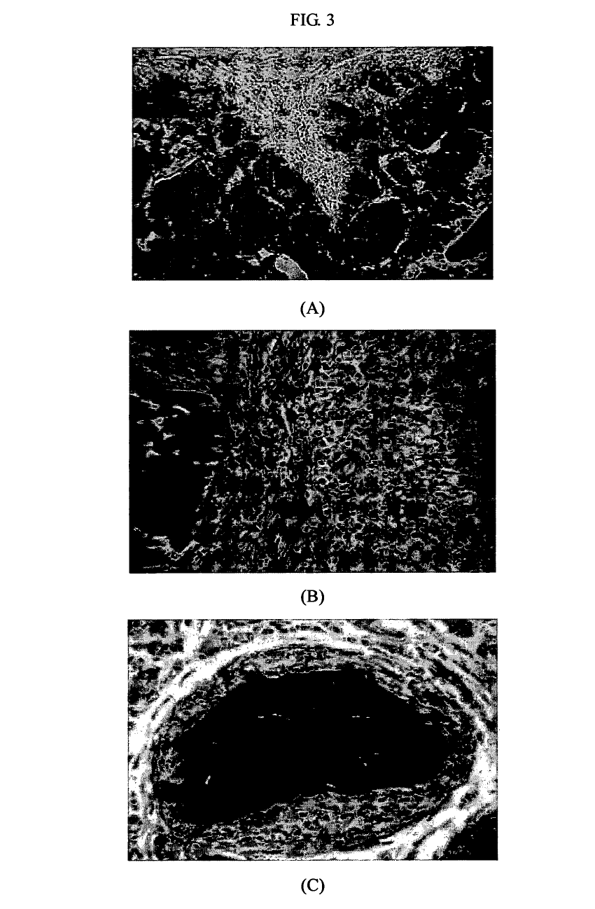

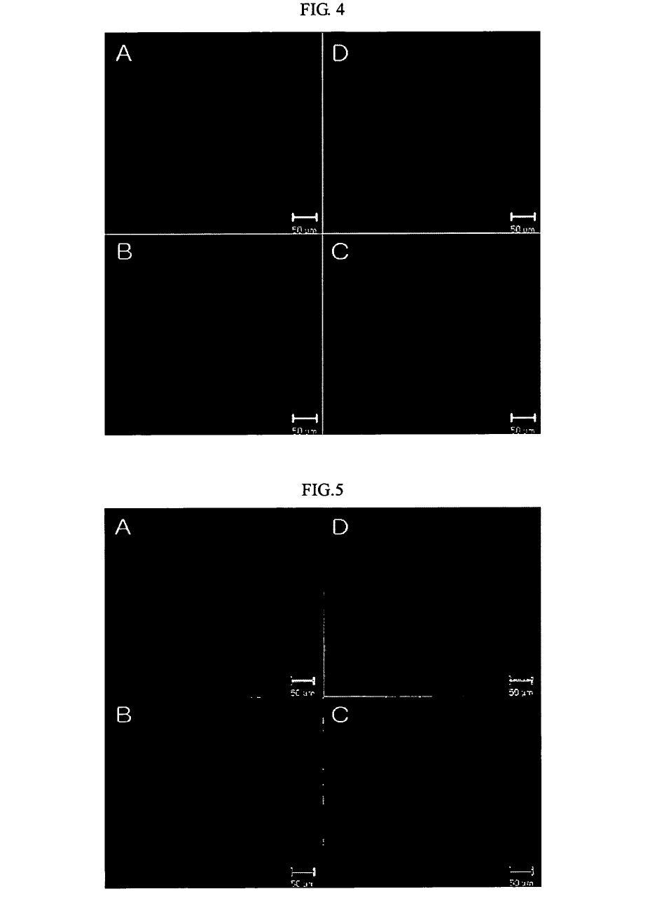

As a result, as shown in FIG. 3, it could be found that a significantly greater amount of osteocytes were newly generated in a group transplanted with a mixture of TCP and cells isolated from canine umbilical cord blood and blood sample from canine fetal heart, compared to both, a group transplanted with TCP alone and a group transplanted with a mixture of TCP and canine spinal cord blood-derived cells. FIG. 3(A) shows an image of tissue biopsy at 1 week after transplanting cells isolated from canine umbilical cord blood and blood sample from canine fetal heart with TCP, and FIG. 3(B) is an image of tissue biopsy at 8 weeks after transplanting cells isolated from canine umbilical cord blood and blood sample from canine fetal heart with TCP, which shows new osteocytes being generated at a high rate around the transplant site. FIG. 3(C) is a photograph of 3(B) taken at 40× magnification and shows osteocytes formed.

### Example 4

### Differentiation of Multipotent Adult Stem Cells Derived from Canine Umbilical Cord Blood and Blood Sample from Canine Fetal Heart into Neural Cells

After the primary culture of multipotent adult stem cell broth derived from canine umbilical cord blood and blood sample from canine fetal heart, obtained in Example 1, P2 cells were collected, followed by seeding and attaching them onto a chamber slide for 2-3 days so as to induce differentiation of the cells. And then, the cells were preincubated in a medium containing 1 mM β-mercaptoethanol for 24 hours and induced to differentiate into neural cells in a medium containing 100M BHA and 1% DMSO for 5 hours.

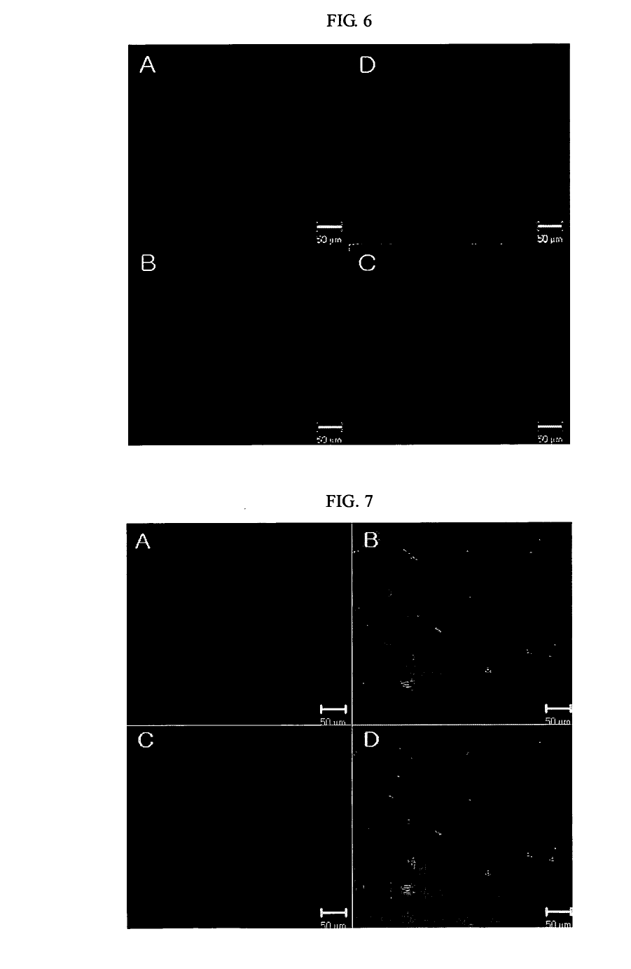

FIG. 4 shows images of the adult stem cells according to the present invention expressing GFAP primary antibody (A: cells expressing GFAP, B: Hoechst staining, C: merger of A and B, D: control). FIG. 5 shows images of the adult stem cells according to the present invention expressing MAP2 primary antibody (A: cells expressing MAP2, B: Hoechst staining, C: merger of A and B, D: control). FIG. 6 shows images of the adult stem cells according to the present invention expressing Tuj1 primary antibody (A: cells expressing Tuj1, B: Hoechst staining, C: merger of A and B, D: control). FIGS. 7 and 8 show images of a negative control in which a secondary antibody is reacted with cells without reaction with a primary antibody (A and E: cells which is reacted with a secondary antibody without reaction with a primary antibody, B and F: DIC images of a confocal microscope, C and G: Hoechst staining of the nuclei of cells, D: merger of A, B, and C, H: merger of E, F, and G). As illustrated in FIGS. 4˜8, it could be found that the cells induced to be differentiated into nerve cells show positive expression of specific neural markers, including GFAP (Glial Fibrillary Acidic Protein), MAP2 (Microtubule-Associated Protein2), and Tuj1.

Although neural cell-related markers were expressed in a control in which neural differentiation was not induced, it has been reported that bone human marrow-derived undifferentiated mesenchymal stem cells express GFAP, MAP2, Tuj1 (Tondreau et al., Differentiation, 72:319-326, 2004). As a result of experimenting with multipotent stem cells isolated from canine umbilical cord blood and blood sample from canine fetal heart, a relatively high level of expression were shown in a group in which neural differentiation was induced.

### Example 5

### Treatment of Spinal Cord Injury Using Multipotent Adult Stem Cells Derived from Canine Umbilical Cord Blood and Blood Sample from Canine Fetal Heart

(1) Cell Transplantation into the Area of Spinal Cord Injury of Spinal Cord Injury Animal Models

After the primary culture of multipotent adult stem cell broth derived from canine umbilical cord blood and blood sample from canine fetal heart, obtained in Example 1, P2 cells were collected. All the animals used in the treatment of spinal cord injury were checked for whether they can be put under anesthesia by examinations of blood, serum and chest X-ray before cell transplantation. After a magnetic resonance imaging test was performed to check the state of spinal cord segments of the area where cells are transplanted, spinal cord lesions of all the animals were exposed by a posterior laminectomy under general anesthesia and spinal cord durotomy was performed. Cell transplantation was performed by directly injecting 1×10⁶˜1×10⁷ cells suspended in 200 μl of sterile physiological saline solution into the exposed spinal cord of an experimental animal under a surgical operating microscope. After cell injection, the incised dura mater was sutured with a hygroscopic thread and the muscles and skins were sutured in a general manner. 4 weeks and 8 weeks after transplantation, Olby score of each animal group was measured and the cell-transplanted area was measured by MRI. The cell-transplanted groups are divided into 4 groups; a control group (C1˜C5) in which physiological saline solution is injected in stead of cells, an experimental group (G1˜G5) in which G-CSF (granulocyte-colony stimulating factor) is injected, an experimental group (UCB G1˜UCB G5) in which G-CSF and canine UCB-derived adult stem cells are injected, and an experimental group (UCB1˜UCB5) in which adult stem cells derived from canine umbilical cord blood and blood sample from canine fetal heart according to the present invention are injected. Each of 4 groups consists of 5 experimental dogs. In general, rats or mice are frequently used as an experimental animal, however, animal studies on dogs use Tarlov score of 0 to 5 to evaluate neurological status of dogs. Olby score suggested by Olby in 1990's is more specified to complement the lack of Tarlov score (5 levels) and it is a method invented, considering to apply to a dog rather than an experimental animal which is widely applicable.

Table 2 is a standard that shows how Olby score described in Example 5 is determined. On the basis of the standard presented in Table 2, the result of measuring Olby scores of the experimental groups at 4 and 8 weeks suggests that three experimental groups (G1˜G5, UCB G1˜G5, and UCB1˜UCB5) have higher scores than the control group (C1˜C5). Among them, the experimental group (UCB1˜UCB5), in which only adult stem cells derived from canine umbilical cord blood and blood sample from canine fetal heart were injected, obtained the highest score compared to other experimental groups.

TABLE 2

| Stage | Score | Neurologic status |
|---|---|---|
| 1 | 0 | no pelvic limb movement and no deep pain sensation |
| | 1 | no pelvic limb movement with deep pain sensation |
| | 2 | no pelvic limb movement but voluntary tail movement |
| 2 | 3 | minimal non-weight-bearing protraction of pelvic limb(movement of one joint) |
| | 4 | non-weight-bearing protraction of pelvic limb with more than one jt. |
| | | involved less than 50% of the time |
| | 5 | non-weight-bearing protraction of pelvic limb with more than one jt. |
| | | involved more than 50% of the time |
| 3 | 6 | weight-bearing protraction of pelvic limb less than 10% of the time |
| | 7 | weight-bearing protraction of pelvic limb 10-50% of the time |
| | 8 | weight-bearing protraction of pelvic limb more than 50% of the time |
| 4 | 9 | weight-bearing protraction 100% of time with reduced strength of pelvic |
| | | limb. mistake >90% time. |
| | 10 | weight-bearing protraction of pelvic limb 100% of time with reduced |
| | | strength. mistake 50-90% of the time. |
| | 11 | weight-bearing protraction of pelvic limb 100% of time with reduced |
| | | strength. mistake <50% of the time. |
| 5 | 12 | ataxic pelvic limb gait with normal strength, but mistakes made >50% |
| | | of time. |
| | 13 | ataxic pelvic limb gait with normal strength, but mistakes made <50% |
| | | of time |
| | 14 | normal pelvic limb gait. |

TABLE 3
Olby score results

| Group | 4 weeks | 8 weeks |
|---|---|---|
| C1(control) | 0 | 0 |
| C2 | 0 | 0 |
| C3 | 0 | 1 |
| C4 | 0 | 0.5 |
| C5 | 0.5 | 1 |
| G1(G-CSF) | 1 | 3 |
| G2 | 4 | 6 |
| G3 | 1 | 2 |
| G4 | 2 | 3 |
| G5 | 1 | 3 |
| UCB G1 (CELL + G-CSF) | 3 | 5 |
| UCB G2 | 3 | 5 |
| UCB G3 | 3 | 5 |
| UCB G4 | 4 | 5 |
| UCB G5 | 5 | 9 |
| UCB1 | 3 | 5 |
| UCB2 | 6 | 9 |
| UCB3 | 6 | 8 |
| UCB4 | 5 | 10 |
| UCB5 | 5 | 6 |

(2) Cell Transplantation into an Experimental Animal with Chronic Spinal Cord Injury

In addition to cell transplantation into the area of spinal cord injury of spinal cord injury animal models, canine UCB-derived adult stem cells were transplanted into the injured spinal cord area of four dogs with chronic spinal cord injury.

Table 4 is a schematic explanation on the state before the transplantation of the inventive multipotent adult stem cells into four dogs (No. 1˜No. 4) in Example 5 and cell transplantation.

TABLE 4

| | | | Durations | | Previous | |
|---|---|---|---|---|---|---|
| | | History & | of | Previous | Examination | Cell amount for |
| No | Signalment | diagnosis | paraplegia | treatment | for this study | transplantation |
| 1 | Shar-pei | Traffic accident, | 13 month | No treatment | Computed | 2 × 10⁶ |
| | 13 month F | L3 vertebral | | | tomography, | |
| | | body fracture | | | SSEP | |
| 2 | CS | IVDD type I | 7 month | Hemi- | Computed | 2 × 10⁶ |
| | 4 yrs M | T13-L1 | | laminectomy | tomography, | |
| | | | | | SSEP | |
| 3 | Pekinese | IVDD type I | 6 month | No treatment | Magnetic | 1 × 10⁶ |
| | 2 yrs NM | T11-T12 | | | resonance | |
| | | | | | image, SSEP | |
| 4 | Malrese | Traffic accident, | 5 month | No treatment | Magnetic | 1 × 10⁶ |
| | 2 yrs F | T13 vertebral | | | resonance | |
| | | body fracture | | | image, SSEP | |

As shown in Table 4, No. 1, a 13-month-old Chinese Shar-Pei female dog was paralyzed for 13 months with severe neural damage due to the third lumbar vertebral body fracture by a traffic accident and was not treated. After computed tomography (CT) and somato sensory evoked potential (SSEP) tests were conducted, 2×10⁴˜2×10⁷ cells were transplanted.

No. 2, a 4-year-old Cocker Spaniel male dog, had hemilaminectomy in the state of complete posterior paralysis due to rupture of the intervertebral disk between the thirteenth thoracic vertebra and the first lumbar vertebra, but was in the state of complete posterior paralysis for 7 months without recovery. After MRI and SSEP tests were performed, 2×10⁴˜2×10⁷ cells were transplanted.

No. 3, a 2-year-old Pekinese male dog was in the state of complete posterior paralysis for 6 months due to rupture of the intervertebral disk between the eleventh and twelfth thoracic vertebra and was not treated. After magnetic resonance image (MRI) and SSEP tests were performed, 1×10⁴˜1×10⁷ cells were transplanted.

No. 4, a 2-year-old Maltese female dog was in the state of complete posterior paralysis for 6 months due to the thirteenth thoracic vertebral body fracture by a traffic accident and was not treated. After MRI and SSEP tests were performed, 1×10⁴˜1×10⁷ cells were transplanted.

As shown in FIG. 5, nerve conduction velocity (NCV) calculated by SSEP was not detected in experimental dogs, dog 1, dog 2, dog 3 and dog 4 before cell transplantation, but in case of dog 1 and dog 3, NCV began to be detected 4 weeks after transplantation. Normal NCV was observed in dog Nos. 1, 2, and 3 16 weeks after transplantation and NCV was slightly detected in dog No. 4 16 weeks after transplantation.

TABLE 5
Results of Nerve Conduction Velocity (NCV)

| | Previous | 2 weeks | 4 weeks | 16 weeks | 32 weeks |
|---|---|---|---|---|---|
| Case No. | examination | (m/s) | (m/s) | (m/s) | (m/s) |
| 1 | NE | NE | 23.6 | 53.1 | 70.8 |
| 2 | NE | NE | NR | 61.1 | NR |
| 3 | NE | NE | 13.7 | 80 | — |
| 4 | NE | NE | NE | 11.5 | — |

(NE: Not Examined, NR: Not Returned)

Moreover, Neurological status and Olby score of each experimental dog 4, 16, and 32 weeks after cell transplantation are schematically summarized in Table 6. Table 6 schematically explains symptoms and clinical changes in four experimental dogs with time after the transplantation of adult stem cells into dog Nos. 1 to 4 in Example 5.

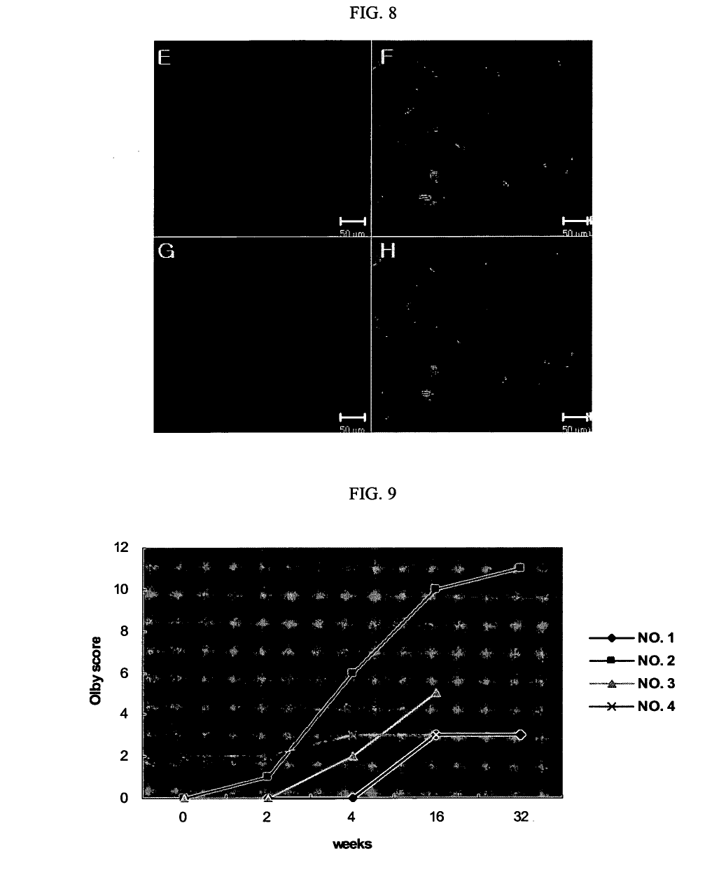

As shown in FIG. 9, three dogs (No. 1, 2, and 3) except the dog 4 showed remarkably high Olby scores at 16 weeks after cell transformation compared to that of initial state of the transplantation.

Also, using a MR system (0.2 Tesla magnet (VET-MR, Esaote, Italy) slice thickness: 5.00 mm, interval 5.00 mm), transverse T2-weighted images were taken on the spinal cord lesion of experimental dogs where canine UCB-derived multipotent adult stem cells were transplanted. Transverse T2-weighted images were obtained at 5 mm-thickness and a pixel matrix of each slide was 256×176. Transverse T2-weighted images were measured with a TR of 3800 msec and a TE of 90 msec, T1-weighted images were measured with a TR of 540 msec and a TE of 26 msec.

TABLE 6

| | | Neurological | | | |
|---|---|---|---|---|---|
| | | status before cell | | | |
| No | Signalment | transplantation | 4 weeks after | 16 weeks after | 32 weeks after |
| 1 | Shar-pei | No Pain sensation | No Pain sensation | Deep pain(+), | All pain sensation, |
| | 13 month F | Olby score | Olby score | Olby score | Olby score |
| | | stage: 1, point: 0 | stage: 1, point: 2 | stage: 1, point: 3 | stage: 2, point: 4 |
| | | Dermatome: | Dermatome: | Dermatome: all | Dermatome: all |
| | | L3 level | L5~6 level | Voluntary | Voluntary |
| | | Involuntary | Voluntary | urination | urination |
| | | urination | urination | Muscle atrophy | Muscle atrophy |
| 2 | CS | No pain sensation | All pain sensation | Weekly | Weakly |
| | 4 yrs M | Olby score | Olby score | ambulatory | ambulatory |
| | | stage: 1, point: 0 | stage: 3, point: 6 | Olby score | Olby score |
| | | Dermatome: | Dermatome: | stage: 4, point: 10 | stage: 4, point: 11 |
| | | L1 level | all level | Dermatome: | (telephone) |
| | | Involuntary | Involuntary | all level | |
| | | urination | urination | Voluntary | |
| | | Muscle atrophy | | urination (+/−) | |
| | | | | Muscle mass | |
| | | | | increased | |
| 3 | Pekinese | No pain sensation | Deep pain | All pain sensation | — |
| | 2 yrs NM | Olby score | sensation | Olby score | |
| | | stage: 1, point: 0 | Olby score | stage: 1, point: 5 | |
| | | Dermatome: | stage: 1, point: 2 | Dermatome: | |
| | | T13 level | Dermatome: | all level | |
| | | Involuntary | L1 level | Involuntary | |
| | | urination | Involuntary | urination | |
| | | | urination | | |
| 4 | Maltese | No pain sensation | No pain sensation | No pain sensation | — |
| | 2 yrs F | Olby score | Olby score | Olby score | — |
| | | stage: 1, point: 2 | stage: 1, point: 2 | stage: 1, point: 2 | |
| | | Dermatome: | Dermatome: | Dermatome: | |
| | | L1 level | L1 level | L3 level | |
| | | Involuntary | Involuntary | Involuntary | |
| | | urination | urination | urination | |
| | | | | Severe muscle | |
| | | | | atrophy | |

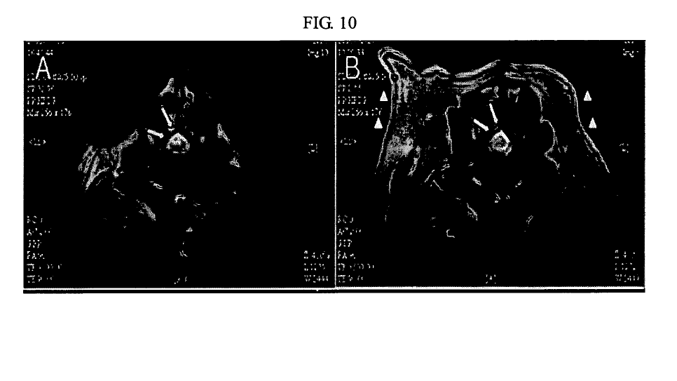

FIG. 10 illustrates the scanned transverse T2-weighted image of the spinal cord lesion of experimental dogs where canine UCB-derived multipotent adult stem cells were transplanted according to the present invention, which is measured by the above described method; including images (A) before cell transplantation and (B) after cell transplantation. In FIG. 10, the arrow indicates the area of spinal cord showing high signal intensity on the T2-weighted image and the arrow head indicates the increased epaxial muscle. Experimental dogs at 16 weeks after stem cell transplantation presented that high signal intensity is decreased around the area of motor neurons of the right posterior funiculus and that the body epaxial muscles on both sides of the spine are increased to some extent. From the above-mentioned results, it could be found that multipotent adult stem cells according to the present invention have remarkable effects on treating canine neural injury.

### INDUSTRIAL APPLICABILITY

As described and proved above in detail, adult stem cells according to the present invention are derived from canine umbilical cord blood, placental blood and blood sample from canine fetal heart. The adult stem cells have characteristics similar to human mesenchymal stem cells as well as show remarkable cell growth at the initial step compared to human UCB-derived mesenchymal stem cells so that the cells are useful to treat canine incurable diseases and difficult-to-cure diseases. Furthermore, the multipotent adult stem cells are effective to treat musculoskeletal diseases and neural diseases due to the ability to differentiate into osteogenic cells and neural cells.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for producing an adult stem cell, wherein the adult stem cell is obtained by culturing eukaryotic cells derived from blood sample from canine fetal heart, and canine umbilical cord blood or placental blood, in FBS-containing medium and show the following characteristics of:
(a) showing positive immunological responses to one or more of antigens selected from the group consisting of MHC class I, CD44(BD) and CD90, and positive or negative immunological responses to CD34, and negative immunological responses to CD45, CD14, CD3, CD4, CD8, CD11c, CD 172a and HLA-DR;
(b) growing adhered to plastic and showing spindle-shaped morphological features; and
(c) having the ability to differentiate into the cells derived from endoderm, ectoderm, and mesoderm.

2. The method for preparing adult stem cells according to claim 1, wherein the medium is DMEM containing 1˜30% FBS.

3. Adult stem cells prepared by the method of claim 1, which show the following characteristics of:
(a) showing positive immunological responses to one or more of antigens selected from the group consisting of MHC class I, CD44(BD) and CD90, and positive or negative immunological responses to CD34, and negative immunological responses to CD45, CD14, CD3, CD4, CD8, CD11c, CD 172a and HLA-DR;
(b) growing adhered to plastic and showing spindle-shaped morphological features; and
(c) having the ability to differentiate into the cells derived from endoderm, ectoderm, and mesoderm.

4. The adult stem cells according to claim 3, wherein the cells derived from mesoderm are osteogenic cells.

5. A method for differentiating adult stem cells into osteogenic cells, the method comprising: mixing the adult stem cells of claim 3 with TCP (Tricalcium phosphate); and transplanting them orthotopically or heterotopically.

6. A cellular therapeutic agent for treating musculoskeletal diseases, containing the adult stem cells of claim 3 as an active ingredient.

7. A cellular therapeutic agent for treating musculoskeletal diseases, which contains the osteogenic cells differentiated by the method of claim 5 as an active ingredient.

8. The adult stem cells according to claim 3, wherein the cells derived from mesoderm are neural cells.

9. The adult stem cells according to claim 8, wherein the neural cells are cerebral nerve cells.

10. A cellular therapeutic agent for treating neural diseases, containing the adult stem cells of claim 3 as an active ingredient.

11. The cellular therapeutic agent according to claim 10, wherein the neural diseases are selected from the group consisting of cerebral infarction, dementia, Parkinson's disease, and spinal cord injury.

12. A cellular therapeutic agent for treating canine difficult-to-cure diseases, containing the adult stem cells of claim 3 as an active ingredient.

13. A method for differentiating the adult stem cells into neural cells, the method comprising the following steps of: (a) preincubating the adult stem cells of claim 3 in a DMEM medium containing β-mercaptoethanol; and (b) treating the preincubated broth with DMSO and BHA so as to induce neural differentiation.

14. A method for treating neural diseases, the method characterized in using the adult stem cells of claim 3 as an active ingredient.

15. A method for treating canine difficult-to-cure diseases, the method characterized in using the adult stem cells of claim 3 as an active ingredient.
